(19) United States
(12) Patent Application Publication
(10) Pub. No.: US 2022/0296830 A1
(43) Pub. Date: Sep. 22, 2022

(54) Ventilation of Airborne Contaminants from a Medical Patient

(71) Applicants: Ivan Sabesky, Winnipeg (CA); Mark Nepon, Winnipeg (CA)

(72) Inventors: Ivan Sabesky, Winnipeg (CA); Mark Nepon, Winnipeg (CA)

(21) Appl. No.: 17/690,395

(22) Filed: Mar. 9, 2022

Related U.S. Application Data
(60) Provisional application No. 63/164,270, filed on Mar. 22, 2021.

(51) Int. Cl. A61M 16/00; A61C 17/06

(52) U.S. Cl. CPC A61M 16/009; A61C 17/06

(57) ABSTRACT

Medical gas or airborne contaminants can be evacuated from a vicinity of a face of a patient using an intake duct defining an intake opening at a single intake location. The single intake opening is supported offset to one side of a face of the patient and an exhaust flow is applied at a flow rate of greater than 200 cubic feet per minute such that a single flow is drawn across the face of the user and into the intake duct. By drawing from only one side of the face of the patient with the prescribed flow rate, the velocity of gases and airborne contaminants flowing towards the intake duct can exceed the forward momentum of gases and/or contaminants expelled by the patient so as to capture substantially all medical gases and/or contaminants in the vicinity of the face of the patient.

This application claims the benefit under 35 U.S.C. 119(e) of U.S. provisional application Ser. No. 63/164,270, filed Mar. 22, 2021.

### FIELD OF THE INVENTION

The present invention relates to the collection of gases, for example medical gases, and airborne contaminants, for example fumes, mists, and pathogens, from the vicinity of the face of a patient.

### BACKGROUND

In the application of anaesthetic or analgesic gases by means of a gas delivery mask to medical and dental patients, it is known for some gas to often escape into the surrounding environment. Also, when treating certain patients, waste gas or bio-contaminants exhaled by the patient or of concern to others in the surrounding area. It is known to be desirable to prevent accumulation of these gases or contaminants in the area immediately surrounding the patient by providing ventilation or means to collect the gases. Many known ventilation or collection devices however interfere with access to the patient's mouth making them unsuitable for certain applications, for example in dentistry.

U.S. Pat. No. 5,513,632 to Nepon et al and U.S. Pat. No. 4,895,172 to Lindkvist each disclose ventilation devices for collecting gases from an area surrounding a patient's face while providing some access to the patient's mouth. In general, these devices are cumbersome and awkward to apply to the face of the patient which results in discomfort of the patient while interfering with access to the patient by persons treating the patient.

U.S. Patent Application Publication No. 2007/0044794 by Sabesky et al discloses a ventilation system comprising an envelope acting as a pillow to support the head of the patient thereon. The envelope has breathable surface portions on each side of the face of the patient for collecting gases therethrough into a hollow interior of the envelope that communicates with a vacuum device. This ventilation system is similar to both patents noted above in that gases are drawn evenly into intake openings at both sides of the face of the patient. This arrangement of intake of gases at both sides of the face of the patient has the effect of an interference between two opposing flows in front of the face of the patient which can reduce the ability of the ventilation system to capture gases and airborne contaminants being expelled by the patient in opposing to the captured flow.

### SUMMARY OF THE INVENTION

According to one aspect of the invention there is provided a method of evacuating medical gas or airborne contaminants from a vicinity of a face of a patient in a patient chair, the method comprising:
providing an intake duct having one or more intake apertures defining an intake opening at a single intake location;
supporting the intake duct adjacent to the patient chair such that the single intake location of the intake duct is offset to one side of a face of the patient; and
applying an exhaust flow to the gas intake duct at a flow rate through the intake duct greater than 200 cubic feet per minute such that a single flow is drawn across the face of the user and into the intake duct at the single intake location.

By providing an intake duct that draws from only one side of the face of the patient with the prescribed flow rate, the velocity of gases and airborne contaminants flowing towards the intake duct for capture exceeds the forward momentum of gases and/or contaminants expelled by the patient so as to capture substantially all medical gases and/or contaminants that may be present in the vicinity of the face of the patient.

More preferably, the exhaust flow is applied to the gas intake duct so that the flow rate through the intake duct is between 200 and 250 cubic feet per minute.

Preferably the intake opening is defined at an open end of the intake duct and the method includes supporting the intake duct to be spaced laterally to one side of the face of the patient such that the open end is directed in a forward direction that the patient is facing at a slope towards an area in front of the face of the patient.

The intake opening is preferably located longitudinally between a chin and a cheekbone of the patient.

The intake duct may be supported such that (i) the single intake location is offset laterally to one side of a face of the patient by a distance of between 2 and 4 inches, or more preferably by a distance of approximately 3 inches.

When the intake opening is defined at an open end of the intake duct, the intake duct may be supported such that the open end is directed in a forward direction that the patient is facing and protrudes forwardly of the face of the patient. For example, the intake opening may protrude forwardly of the face of the patient by a distance between 2 and 4 inches, and more preferably by a distance of approximately 3 inches.

When a terminal velocity distance is defined as a distance forward of the mouth of the user at which point expelled gases and contaminants from the mouth of the user reach a terminal velocity, preferably the open end of the intake duct protrudes forwardly of the face of the patient by a distance exceeding said terminal velocity distance.

The intake duct may have a diameter between two and six inches, and more preferably between 3 and 4 inches.

The intake duct may be supported on the chair using a supporting frame in which the supporting frame includes a pivot assembly defining a longitudinal pivot axis about which the intake duct is pivotal between a first position offset laterally to a first side of the face of the patient and a second offset laterally to a second side of the face of the patient.

The support frame may further comprise a pivot clamp arranged to selectively fix the pivot assembly immovably about the longitudinal pivot axis so as to retain the intake duct in either selected one of the first position or the second position.

The support frame may further comprise a swivel assembly coupling the intake duct relative to the chair for pivotal movement about a tilt axis that is oriented laterally in each of the first position and the second position so that a longitudinal position of the intake location may be adjusted relative to the face of the patient by pivoting the intake duct about the tilt axis.

The intake duct may be supported on the chair using a supporting frame that includes a chair clamp by releasably clamping the chair clamp onto the patient chair.

When the patient chair includes a back portion supporting a back of the user thereon and a headrest portion joined to the back portion by a post in which the headrest portion supports a head of the user thereon, the intake duct may be supported on the chair by releasably clamping the chair clamp onto the post of the headrest portion.

The intake duct may include an end portion locating the intake opening therein in which the end portion is flexible and has a shape memory arranged to retain a shape of the end portion upon being flexed by a user, so that a position of the intake location can be adjusted relative to the face of the patient by flexing of the end portion of the intake duct.

### BRIEF DESCRIPTION OF THE DRAWINGS

One embodiment of the invention will now be described in conjunction with the accompanying drawings in which:

FIG. 1 is a schematic end view of the ventilation system supported on a patient chair;

FIG. 2 is a schematic side view of the ventilation system supported on the patient chair;

FIG. 3 is a side view of a pivot assembly of the ventilation system for displacing the intake duct between left and right sides of the patient chair; and

FIG. 4 is sectional view of the intake duct of the ventilation system.

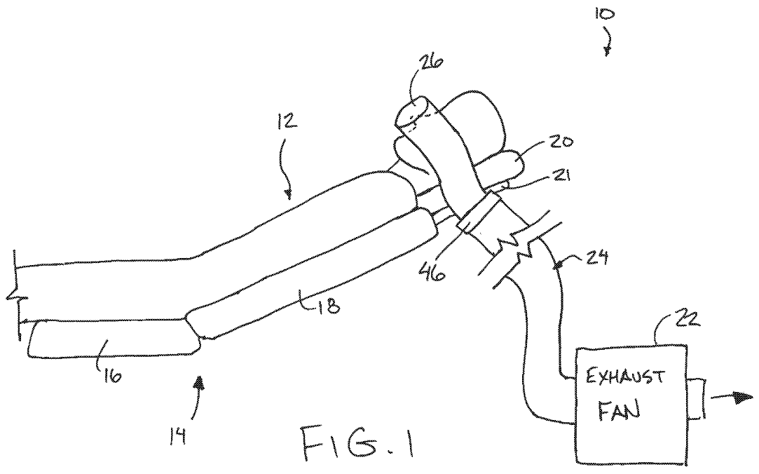

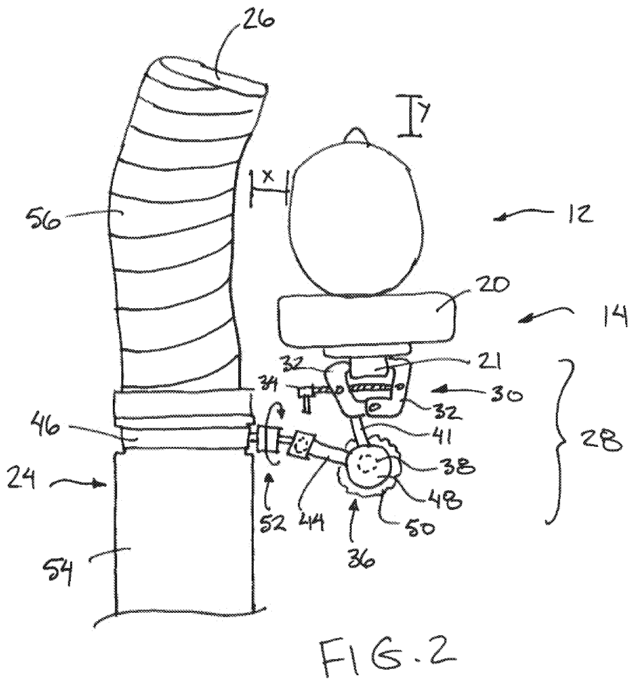

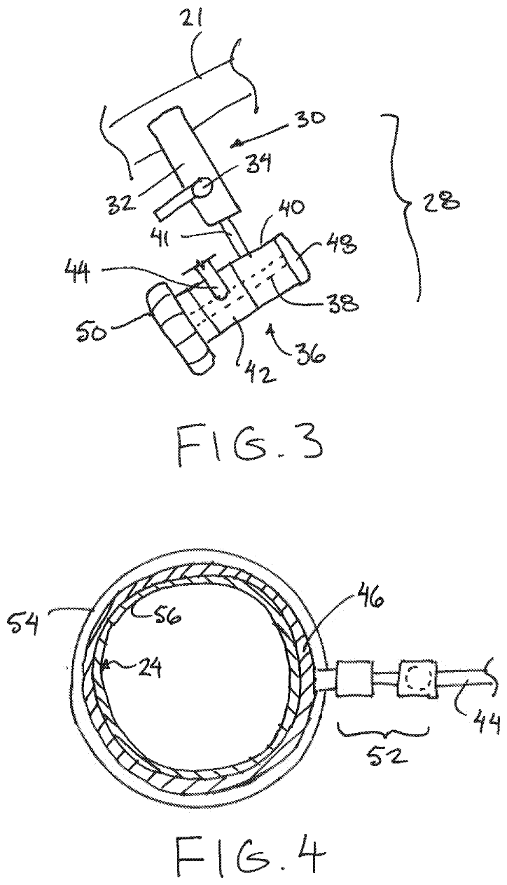

In the drawings like characters of reference indicate corresponding parts in the different figures.

### DETAILED DESCRIPTION

Referring to the accompanying figures there is illustrated a ventilation system 10 for collection of medical gases and/or airborne contaminants, including fumes, mists, and pathogens, from the vicinity of the face of a patient 12 sitting in a medical patient chair 14.

The ventilation system 10 may be incorporated into the patient chair 14 or may be provided as an add-on accessory which can be retrofitted onto an existing medical chair for supporting patients thereon. According to the illustrated embodiment, the system 10 can be retrofitted onto an existing medical chair. In this example, the chair includes a seat bottom portion 16 upon which a patient can be seated, a seat back portion 18 supporting the back of the patient thereon, and a headrest portion 20 at the top end of the seat back portion for supporting the head of the user thereon. The headrest portion 20 may be joined to the seat back portion 18 so as to be adjustably carried on a supporting post 21 of the chair.

The chair is typically adjustable to allow the patient to be supported thereon through a range of positions from an upright seated position to a flat supine position, including a range of intermediate positions therebetween in which the back portion extends at an upward slope. Regardless of the orientation of the back portion, the forward direction as referred to herein generally corresponds to the direction that the patient is facing regardless of whether the patient is in an upright seated position or a reclined supine position.

The system 10 generally comprises (i) an exhaust fan 22, or other suitable vacuum device, arranged to provide a source of vacuum pressure or exhaust flow at a sustained flow rate, (ii) an intake duct 24 defining an elongate exhaust passage therein between an open end 26 defining an intake opening and an opposing end fed into the vacuum device 22, and (iii) a supporting frame 28 for adjustably supporting the intake duct on or adjacent to the patient chair.

In some embodiments, one or more intake apertures may collectively define the intake opening, however the one or more intake apertures in each instance define the intake opening at a single intake location on the intake duct such that application of an exhaust flow to the intake duct by the exhaust fan 22 or vacuum device generates a flow rate through the intake duct that produces a single flow drawn across the face of the user and into the intake duct at the single intake location thereof. The exhaust fan 22 is vented externally to the outdoors.

The supporting frame 28 in the illustrated embodiment includes a chair clamp 30 arranged to be releasably clamped onto the post 21 that supports the headrest portion relative to the back portion of the patient chair. The chair clamp 30 includes two jaws 32 defining clamping surfaces at the outer ends of the jaws which face one another to clamp the post 21 therebetween. The opposing inner ends of the jaws are pivotally connected to one another about a jaw axis that is parallel to the longitudinal direction of the chair and corresponding to the orientation of the post 21. A clamping screw 34 is operatively connected between the two jaws at an intermediate location on each jaw so that manually tightening the clamping screw using a handle on one end of the clamping screw functions to urge the jaws 32 towards one another at the outer ends thereof for clamping the post of the chair therebetween.

A pivot assembly 36 of the supporting frame comprises a shaft 38 having an axis that is parallel to the longitudinal direction of the patient chair and corresponding to a direction of measurement of height of the patient. A first collar 40 is rotatably supported about the pivot shaft and is connected in fixed relation to one of the jaws 32 of the chair clamp 30 by a first stem 41 extending radially from the first collar. A second collar 42 is rotatably supported about the pivot shaft 38 adjacent to the first collar so as to allow relative rotation between the collars about a longitudinal pivot axis of the pivot assembly. A second stem 44 protrudes radially out from the second collar 42 for connection to a supporting collar 46 that is circumferentially clamped about the intake duct 24.

The pivot assembly 36 further includes a pivot clamp defined by a head 48 supported at one end of the pivot shaft and a nut 50 threaded onto the opposing end of the pivot shaft. A manual handgrip is provided on the nut 50 such that the nut can be manually rotated relative to the pivot shaft to selectively clamp the first and second collars between the head 48 and nut 50 of the pivot clamp. When tightened, the first and second collars can be fixed relative to one another by friction resulting from clamping in the axial direction of the pivot shaft. The pivot clamp thus allows the pivot assembly to be immovably fixed at a selected position. The pivot assembly allows the support collar 46 supporting the intake duct thereon to be pivoted through a range of 180 degrees or more between a first position in which the intake duct is laterally offset to the left side of the patient in the chair and a second position in which the intake duct is laterally offset to the opposing right side of the patient in the chair.

A swivel assembly 52 is connected between the second stem 44 and the support collar 46 so as to enable some adjustment of the support collar and the intake duct supported thereon about a tilt axis that is oriented in a lateral direction in either one of the first or second positions of the pivot assembly. The swivel assembly may comprise a ball and socket arrangement in which the ball is friction fit within the socket so as to enable the support collar to be pivoted in any direction relative to the pivot assembly 36 while having sufficient friction to retain the orientation of the support collar and the intake duct relative to the pivot assembly in the selected orientation of the swivel assembly.

The intake duct includes a main portion 54 in communication between the vacuum device 22 and the support collar 46 of the support frame 28. The duct further includes an end portion 56 protruding beyond the support collar 46 to the open end 26 of the intake duct. The end portion of the duct has a circular cross-section and is formed of a pliable plastic material that includes an embedded helical support wire therein. The plastic boundary material of the duct is readily pliable and the helical support wire is also flexible to allow the end portion of the duct to be reoriented and repositioned as desired. The material of the support wire of the duct includes shape memory such that it is capable of retaining a deflected position when flexed or deflected manually to vary the shape by the user.

In use, the intake duct is initially attached to the patient chair using the chair clamp 30 of the support frame. Alternatively, the intake duct may already be supported on the chair when incorporated integrally into the structure of the chair. The user initially selects whether the intake location defined by the intake opening at the open end 26 of the duct is to be positioned offset laterally to the left side or the right side of the patient based upon where the medical professional would prefer to access the patient. Once the selected first or second position of the pivot assembly 36 has been determined, the pivot clamp can be tightened to fix the pivot assembly immovably at the selected position.

Once the left or right side of the patient has been selected, further adjustment of the position of the intake duct can be adjusted by pivoting the end portion of the intake duct about the tilt axis of the swivel assembly 52 which allows pivotal movement through a range of between 20 and 30 degrees for example. Pivoting about the tilt axis allows the open end of the intake duct to be displaced generally in the longitudinal direction of the patient corresponding to a direction that the height of a person would be measured. The swivel further allows the intake duct to extend forwardly relative to the patient at a slope in the longitudinal direction towards the top of the head of the patient. Finally, the end portion of the intake duct itself can be flexed into position relative to the patient to finally adjust the open end of the intake duct laterally towards and away from the cheek of the user and longitudinally relative to the height of the patient. The flexing of the duct allows deflection of between 20 and 30 degrees in any direction from a neutral linear position or orientation of the duct.

The duct may have varying diameters, however a duct in the range of 2 inches to 6 inches in diameter is preferred. More preferably the intake duct has a diameter between 3 and 4 inches. In this instance, the exhaust fan is operated to have a flow rate which exceeds 200 cubic feet per minute, and more preferably which is between 200 and 250 cubic feet per minute.

In the preferred operation of the system, the open end of the intake duct defining the intake opening at a single intake location is preferably offset laterally by a distance X of between 2 and 4 inches, and more preferably approximately 3 inches from the corresponding cheek of the patient. In the longitudinal direction, the open end of the duct is preferably located between the chin and cheekbone of the patient.

The duct is typically positioned such that the open and protrudes forwardly beyond the mouth or face of the patient by a distance Y of between 2 and 4 inches, and more preferably near 3 inches. A terminal velocity distance may be defined as the distance Y extending forwardly from the mouth of the user at which point expelled gases and contaminants from the mouth of the user reach a terminal velocity and/or begin to decelerate. The open end of the duct is preferably supported such that it is located to protrude forwardly of the face of the patient by a corresponding distance which meets or exceeds the defined terminal velocity distance.

Since various modifications can be made in my invention as herein above described, and many apparently widely different embodiments of same made, it is intended that all matter contained in the accompanying specification shall be interpreted as illustrative only and not in a limiting sense.

1. A method of evacuating medical gas or airborne contaminants from a vicinity of a face of a patient in a patient chair, the method comprising:
providing an intake duct having one or more intake apertures defining an intake opening at a single intake location;
supporting the intake duct adjacent to the patient chair such that the single intake location of the intake duct is offset to one side of a face of the patient; and
applying an exhaust flow to the intake duct at a flow rate through the intake duct greater than 200 cubic feet per minute such that a single flow is drawn across the face of the user and into the intake duct at the single intake location.

2. The method according to claim 1 including applying the exhaust flow to the gas intake duct so that the flow rate through the intake duct is between 200 and 250 cubic feet per minute.

3. The method according to claim 1 wherein the intake opening is defined at an open end of the intake duct and wherein the method includes supporting the intake duct to be spaced laterally to one side of the face of the patient such that the open end is directed in a forward direction that the patient is facing at a slope towards an area in front of the face of the patient.

4. The method according to claim 1 further comprising locating the intake opening longitudinally between a chin and a cheekbone of the patient.

5. The method according to claim 1 further comprising supporting the intake duct such that the single intake location is offset laterally to one side of a face of the patient by a distance of between 2 and 4 inches.

6. The method according to claim 1 further comprising supporting the intake duct such that the single intake location is offset laterally to one side of a face of the patient by a distance of approximately 3 inches.

7. The method according to claim 1 wherein the intake opening is defined at an open end of the intake duct and wherein the method includes supporting the intake duct such that the open end is directed in a forward direction that the patient is facing and protrudes forwardly of the face of the patient.

8. The method according to claim 7 including supporting the open end of the intake duct to protrude forwardly of the face of the patient by a distance between 2 and 4 inches.

9. The method according to claim 7 including supporting the open end of the intake duct to protrude forwardly of the face of the patient by a distance of approximately 3 inches.

10. The method according to claim 7 further comprising supporting the open end of the intake duct to protrude forwardly of the face of the patient by a distance exceeding a terminal velocity distance, the terminal velocity distance being defined as a distance forward of the mouth of the user at which point expelled gases and contaminants from the mouth of the user reach a terminal velocity.

11. The method according to claim 1 wherein the intake duct has a diameter between two and six inches.

12. The method according to claim 1 further wherein the intake duct has a diameter between 3 and 4 inches.

13. The method according to claim 1 further comprising supporting the intake duct on the chair using a supporting frame in which the supporting frame includes a pivot assembly defining a longitudinal pivot axis about which the intake duct is pivotal between a first position offset laterally to a first side of the face of the patient and a second offset laterally to a second side of the face of the patient.

14. The method according to claim 13 wherein the support frame further comprises a pivot clamp arranged to selectively fix the pivot assembly immovably about the longitudinal pivot axis so as to retain the intake duct in either selected one of the first position or the second position.

15. The method according to claim 13 wherein the support frame further comprises a swivel assembly coupling the intake duct relative to the chair for pivotal movement about a tilt axis that is oriented laterally in each of the first position and the second position and wherein the method includes adjusting a longitudinal position of the intake location relative to the face of the patient by pivoting the intake duct about the tilt axis.

16. The method according to claim 1 further comprising supporting the intake duct on the chair using a supporting frame that includes a chair clamp by releasably clamping the chair clamp onto the patient chair.

17. The method according to claim 16 wherein the patient chair includes a back portion supporting a back of the user thereon and a headrest portion joined to the back portion by a post, the headrest portion supporting a head of the user thereon, the method comprising supporting the intake duct on the chair by releasably clamping the chair clamp onto the post of the headrest portion.

18. The method according to claim 1 wherein the intake duct includes an end portion locating the intake opening therein, the end portion being flexible and having a shape memory arranged to retain a shape of the end portion upon being flexed by a user, the method including adjusting a position of the intake location relative to the face of the patient by flexing of the end portion of the intake duct.
